(19) United States
(12) Patent Application Publication
(10) Pub. No.: US 2021/0128251 A1
(43) Pub. Date: May 6, 2021

(54) MEDICAL-TECHNICAL INSTRUMENTATION AND METHOD

(71) Applicant: Aesculap AG, Tuttlingen (DE)

(72) Inventors: Georg HETTICH, Freiburg (DE); Ronja SCHIERJOTT, Tuttlingen (DE); Allan MAAS, Konstanz (DE); Christoph SCHILLING, Balgheim (DE)

(21) Appl. No.: 17/144,376

(22) Filed: Jan. 8, 2021

Related U.S. Application Data
(63) Continuation of application No. PCT/EP2019/068198, filed on Jul. 8, 2019.

(30) Foreign Application Priority Data
Jul. 9, 2018 (DE) 10 2018 116 558.7

(51) Int. Cl. A61B 34/20; A61B 34/00; A61B 90/00

(52) U.S. Cl. CPC A61B 34/20; A61B 34/25; A61B 90/39; A61B 2090/365; A61B 2034/2046; A61B 2090/367; A61B 2090/3762; A61B 90/37

(57) ABSTRACT

Medical-technical instrumentation and a medical-technical method that utilize a planning data set created for a surgeon on the basis of an actual state data set of a bone that is considered to be defective. The planning data set can be used intraoperatively for improving the surgery outcome.

### CROSS-REFERENCE TO RELATED APPLICATIONS

This application is a continuation of international application number PCT/EP2019/068198, filed on Jul. 8, 2019, and claims the benefit of German application number 10 2018 116 558.7, filed Jul. 9, 2018, which are incorporated herein by reference in their entirety and for all purposes.

### FIELD

The present disclosure relates to a medical-technical instrumentation for assisting a surgeon in the treatment of a bone, in particular the human pelvic bone, and a medical-technical method.

The present disclosure relates in particular to an instrumentation and a method for use in the treatment of a bone having high-grade defects, for example in revision procedures in hip endoprosthetics, which are used in the following as an example of one application. However, the present disclosure is not limited to this application, it is also suitable for use outside of revision procedures in which, in particular, there are poor preconditions regarding the presence and/or substance or quality of the bone from the outset.

### BACKGROUND

In the conventional case, the revision of a hip implant, in particular the replacement of an artificial acetabulum, in the presence of higher-grade bone defects is still a great challenge for a surgeon. For example, when bone substance is lacking in the region of the acetabulum, the surgeon does not have an orientation aid for positioning the revision implant. The surgeon can be assisted in the revision procedure with the help of comparisons of the abnormal side of the pelvic bone with the healthy side of the pelvic bone, for example in x-ray images. The surgeon's comprehensive experience in revision procedures is helpful and advantageous. However, in practice it occurs that even experienced surgeons lack the necessary orientation due to the lack of anatomical landmarks on the pelvic bone, thus negatively affecting the implantation outcome. For example, the application of the conventional “sloof technique” often leads to a too far lateral position of the revision implant.

When inserting an artificial acetabulum, the alignment thereof typically takes place in relation to a reference plane, in particular the anterior pelvic plane of the patient. For example an inclination angle and an anteversion angle of the acetabulum are used as a measure for the orientation. In the alignment of the acetabulum relative to the pelvis, however, it should be kept in mind that the anterior pelvic plane may be inclined relative to the frontal plane—a vertical plane when the patient is standing, a horizontal plane when the patient is lying down. This is referred to as pelvic tilt. The pelvic tilts in a standing patient and in a patient lying down differ from each other, though, due to the load-bearing situation. The pelvis-femur system should not be considered in isolation, rather, in particular, muscles and tendons have an influence on the musculoskeletal system. When the acetabulum is implanted in a patient lying down, incorrect loading, loosening of the implant and/or mobility restrictions may occur if the pelvic tilt is not taken into account and the difference in pelvic tilt when the patient is standing and when the patient is lying down is not taken into account.

In DE 10 2013 111 808 A1 an instrumentation and a method for simulation purposes in hip endoprosthetics is described, with which the influence of the pelvic tilt can be simulated. An instrumentation for determining the pelvic tilt is described in DE 10 2014 107 832 A1.

DE 10 2013 219 470 A1 describes a method for preoperatively planning a surgical procedure and a computing system. In this case, with a bone fractured into a plurality of segments, physical replicas of the bone fragments are produced, which are repositioned to an anatomically correct bone model. Relative linear offsets and rotations of the fragments are determined. In dependence thereon, a surgery plan is created in which the relative offsets and rotations are predetermined for repositioning the bone fragments.

US 2018/0185100 A1 describes an embodiment of a surgical navigation system.

An object underlying the present disclosure is to provide an instrumentation which assists the surgeon and a method for assisting the surgeon in the treatment of the bone with a view to a better outcome, in particular in a revision surgery in hip endoprosthetics.

### SUMMARY

In a first aspect of the present disclosure, a medical-technical instrumentation comprises at least one data processing device, which is configured and programmed to create an actual state data set of a bone that is considered to be defective of a patient, in particular the human pelvic bone, based on examination data;

to create computationally a healthy state data set of the bone on the basis of the actual state data set;
to create a planning data set of the bone on the basis of the healthy state data set, the anatomical characteristics (in particular the pelvic tilt when standing) and instructions of the surgeon regarding a treatment of the bone, which instructions are providable by way of an input device of the instrumentation, wherein the planning data set in particular comprises information about characteristic anatomical features of the bone;
to display the planning data set on a display device of the instrumentation;
wherein the instrumentation comprises a medical-technical navigation system and a marking device for defining a reference, which marking device is detectable by said navigation system and is fixed or fixable to the bone, wherein location and/or position data of the marking device are providable by the navigation system;
and wherein the planning data set is displayable by at least one data processing device on the display device in spatial relationship with the bone, associating characteristic landmarks of the bone with corresponding characteristic landmarks in the planning data set.

In a second aspect of the present disclosure, a medical-technical method is provided, in which:

an actual state data set of a bone of a patient, in particular the human pelvic bone, which is considered to be defective, is created based on examination data;
a healthy state data set of the bone is created computationally on the basis of the actual state data set;
a planning data set of the bone is created on the basis of the healthy state data set, the anatomical characteristics and instructions of the surgeon regarding a treatment of the bone, which instructions are providable by way of an input device of the instrumentation, wherein the planning data set in particular comprises information about characteristic anatomical features of the bone;
wherein the planning data set is displayable on a display device;
wherein a marking device for defining a reference being fixed to the bone is detected by means of a medical-technical navigation system, and location and/or position data of the marking device are provided by the navigation system;
and the planning data set is displayed on the display device in spatial relationship with the bone, associating characteristic landmarks of the bone with corresponding characteristic landmarks in the planning data set.

### BRIEF DESCRIPTION OF THE DRAWING FIGURES

The foregoing summary and the following description may be better understood in conjunction with the drawing figures, of which:

FIG. 1: shows a schematic perspective depiction of an instrumentation in accordance with the present disclosure in use by a surgeon on a patient with a bone to be treated, said bone presently being the pelvic bone;

FIG. 2: shows a graphic representation of the image content of an actual state data set of the pelvic bone, seen from lateral;

FIG. 3: shows a graphic representation of the image content of a healthy state data set of the pelvic bone, seen from lateral;

FIG. 4: shows a graphic representation of the image content of a planning data set of the pelvic bone, seen from lateral;

FIG. 5: shows a schematic partial depiction of a pair of data glasses for the surgeon, said glasses having a display device, wherein image content of the planning data set is displayed;

FIG. 6: shows a depiction corresponding to FIG. 5, with a different depiction of the image content of the planning data set; and

FIG. 7: shows a depiction corresponding to FIG. 1 with a further preferred embodiment of the instrumentation in accordance with the present disclosure.

### DETAILED DESCRIPTION

Although the present disclosure contains illustrations and descriptions of specific embodiments, the present disclosure is not intended to be limited to the details shown. Rather, various modifications may be made in the details within the scope and range of equivalents and without departing from the present disclosure.

The present disclosure relates to a medical-technical instrumentation comprising at least one data processing device, which is configured and programmed to create an actual state data set of a bone that is considered to be defective of a patient, in particular the human pelvic bone, based on examination data;

to create computationally a healthy state data set of the bone on the basis of the actual state data set;
to create a planning data set of the bone on the basis of the healthy state data set, the anatomical characteristics (in particular the pelvic tilt when standing) and instructions of the surgeon regarding a treatment of the bone, which instructions are providable by way of an input device of the instrumentation, wherein the planning data set in particular comprises information about characteristic anatomical features of the bone;
to display the planning data set on a display device of the instrumentation;
wherein the instrumentation comprises a medical-technical navigation system and a marking device for defining a reference, which marking device is detectable by said navigation system and is fixed or fixable to the bone, wherein location and/or position data of the marking device are providable by the navigation system;
and wherein the planning data set is displayable by at least one data processing device on the display device in spatial relationship with the bone, associating characteristic landmarks of the bone with corresponding characteristic landmarks in the planning data set.

In the instrumentation in accordance with the present disclosure, the advantage for the surgeon is in particular that, unlike conventional scenarios, a linking of a preoperative planning with an intraoperative application takes place and the surgeon can thereby be aided intraoperatively using information that has gone into the planning of the procedure, for example by way of a workflow implemented on the software side. In the field of intraoperative procedure, there is the advantage, in particular, that the planning of the procedure can be checked with a view to the improvement thereof and can be adapted if necessary.

The instrumentation comprises at least one data processing device, wherein a plurality thereof may be provided, which may be used in different planning steps. A data processing device is configured and programmed such that it uses examination data of the bone to be treated, for example on the basis of X-ray images and/or CT images. An actual state data set of the “bad” bone is created, wherein data sets, in the present case, preferably comprise a three-dimensional representation of the bone. The data processing unit can computationally create a healthy state data set of the bone. For example statistical shape models may be used here. Creating the actual state data set individually to the patient instead of using a generic standard data set for the bone is advantageous. A planning data set can subsequently be created, taking into account planning instructions of the surgeon. Said planning data set may in particular comprise clinically relevant information, in the case of hip endoprosthetics, for example, the location of the plane of the acetabulum, the rotation center, and/or the axis of the acetabulum. Alternatively or in addition, the planning data set may advantageously have suggestions and/or indications for the surgeon for the procedure, which will be described in the following. A reference can be created on the bone intraoperatively by means of the marking device, which is detectable by the navigation system in a manner known per se. Characteristic landmarks of the bone may be associated with characteristic landmarks of the planning data set. This makes it possible to display on the display device of the instrumentation the visual information of the planning data set in defined spatial relationship to the bone. The surgeon thus obtains a visual aid in the sense of an augmented reality (AR). It is hereby possible, for example, to optically superimpose the computed representation of the bone in the planning data set over a depiction of the real bone on the display device. By tracking the marking device by means of the navigation system, changes in the location of the bone in the surgical system can be determined. Because the bone is known in the reference system of the marking device with respect to location and geometry, planning data can remain computationally “pinned” to the bone when the bone moves and corresponding visual information can preferably be made permanently available to the surgeon on the display device.

The actual state data set advantageously comprises a three-dimensional representation of the bone.

In a preferred embodiment, the instrumentation may comprise at least one imaging device. For example the actual state data set may be createable using the at least one device. Alternatively or in addition, by means of the at least one device, an auxiliary data set may be createable with which the association of the characteristic landmarks is made by the data processing device. It may thereby advantageously be possible for the data processing device to register the marking device relative to the actual state data set and/or the planning data set.

The at least one imaging device is or comprises e.g. an X-ray apparatus or CT apparatus.

Provision may be made for the auxiliary data set to be or comprise an X-ray image of the bone and the marking device, and for the data processing device to superimpose the X-ray image over the actual state data set and/or the planning data set. The association of characteristic landmarks resulting therefrom ideally takes place automatically and intraoperatively by means of the data processing device.

The association of the planning data set with the bone of the patient may, for example, be made by an X-ray image being created intraoperatively. For this purpose, for example a C-arc X-ray apparatus is used, with which a two-dimensional X-ray image is created. The X-ray image depicts the bone, in particular the pelvic bone, and the marking device. The data processing device can superimpose the X-ray image over the actual state data set. Because the planning data set is based on the latter, a superimposition of the X-ray image with the planning data set may advantageously also be performed. Upon the superimposition, optimization algorithms may be used by the data processing device, which e.g. superimpose brightness values of the X-ray image with those of the three-dimensional model of the bone.

In a further step, the orientation of the marking device relative to the model of the bone can be determined. Based on the X-ray image and taking the foreknowledge of the three-dimensional anatomy of the bone into account, a three-dimensional reference system between the marking device and the bone can be created. As a result of this referencing of the marking device relative to the model of the bone, the navigation system can determine the location and/or orientation of the bone on the basis of the position referenced by the marking device. Upon a movement of the patient during the procedure, the image information of the planning data set can be moved together therewith, because the referencing relates to the marking device that is fixed relative to the bone. The planning data set is “pinned” to the bone, so to speak.

Provision may be made for the marking device to be fixed or fixable directly or indirectly to the bone. A fixing of the marking device to the bone may be non-invasive or invasive.

The depiction of the image content of the planning data set on the display device preferably takes place in real time relative to the patient in order to assist the surgeon in the procedure.

It is favorable if the navigation system comprises at least one camera for detecting the bone and images corresponding thereto are displayable on the display device in combination with the display of the planning data set. The image content of the planning data set can in this way be optically associated with images of the bone, for example overlaid thereon or integrated therein. Planning information, on the basis of which the surgeon, in particular, can compare the healthy state of the bone with e.g. clinically relevant parameters in relation to the ill actual state of the bone, can in this way be perceived particularly intuitively by the surgeon.

The navigation system is or comprises, e.g., a head-mounted display, which comprises the display device. This offers the advantage that the image content of the planning data set is displayed in the surgeon's field of vision, without him/her having to look away from the surgical field to a spatially remotely positioned display device. Said image content may be displayed such that it is located visually above the bone, for example, while associating corresponding characteristic landmarks. The camera mentioned above may, for example, be dispensed with in this case.

The same advantages can be achieved in a preferred embodiment by the navigation system being or comprising a pair of data glasses, which comprise the display device.

It is favorable if a marking device, which is detectable by the navigation system, is arranged on the display or on the data glasses, the movement of said marking device being tracked in space, the planning data set being displayed in dependence on the location and/or orientation of the display or data glasses. In this way, the association of the image content of the planning data set with the bone can be made when the surgeon moves with the display or the glasses. The movement can be tracked by the navigation system. Information corresponding thereto can be transmitted to the data processing device. Depending on the viewing direction of the surgeon, the image content can be adapted in such a way that it, in particular, corresponds with that image content of the real bone that the surgeon sees (e.g. viewing direction toward the bone, portion of the bone etc.).

It is advantageous if the display or the data glasses form the navigation system for detecting the marking device on the bone. A separate medical-technical navigation system may be dispensed with in this way. A measuring system of the navigation system for generating the location and/or position data of the marking device may be physically integrated into the display or the data glasses. Alternatively or in addition, at least one data processing device is preferably integrated into the display or the data glasses.

In a preferred embodiment of the instrumentation, provision may be made for said instrumentation to comprise a hand-holdable integrated navigation system, which comprises at least one data processing device and a display device, for example in the form of a smartphone or tablet computer. “Integrated” may be understood to mean that the data processing device and the display device are arranged in a common housing. The instrumentation preferably further has a camera for detecting the marking device and the bone. Corresponding images can be displayed on the display device and be enriched with the image content of the planning data set. The measuring system for determining the location and/or position data of the marking device is preferably also integrated into the hand-holdable navigation system.

It is advantageous if the data processing device is configured and programmed to determine deviations between the actual state data set and the healthy state data set.

It is hereby advantageous in particular if the data processing device is configured and programmed to classify deviations with regard to at least one of the following:
bone loss of the defective bone compared to the reconstructed healthy bone;
bone growth of the defective bone compared to the reconstructed healthy bone;
material different from bone on the defective bone, for example bone replacement material or bone cement;
the amount or the degree of deviation, for example the size of the bone defect or its extent in at least one spatial direction;
the location of the deviation, for example the position of the bone defect on the bone.

The data processing device is advantageously configured and programmed to perform a classification on the bone in segments. This can presently be understood, in particular, as the bone being able to be computationally segmented, for example into clinically relevant sectors. The clinical relevance may depend on the procedure to be performed, wherein the sectors may have a higher or lower relevance in different procedures. The segmentation of the classification offers the surgeon the advantage of, for example, not getting lost in details in planning, but rather proceeding in a structured and systematic manner.

In a preferred embodiment, the data processing device is configured and programmed to, in dependence on the classification, provide indications in particular on the display device and/or to add indications to the planning data set for performing the procedure. This assists the surgeon in the planning and/or performance of the surgery.

Indications may relate to implantation technique, for example the type of implantation, a suggestion for the bone structure (for example the use of porous metal foams, of bone replacement material, bone cement, or the removal of bone at another location and introducing it into the bone to be treated).

Alternatively or in addition, the indications may relate e.g. to the implant selection, for example the type of implant and/or the size of the implant.

As mentioned at the outset, the bone may be, in particular, the pelvic bone. The possibility of the patient having a pelvic tilt in which the anterior pelvic plane is inclined relative to the frontal plane has also already been discussed.

It is favorable if the planning data set in a treatment of the pelvic bone comprises information about the pelvic tilt when the patient is standing and/or lying down. By means of this additional information, the surgeon can be instructed with a view to an improved implantation outcome. It may be advantageous, in particular, for the different orientation of the pelvic bone between the patient standing and the patient lying down to be taken into account in the planning data set.

The pelvic tilt, for example when the patient is standing, can be determined, in particular preoperatively, for example by means of an imaging device. For example, an X-ray image may be combined with a CT image. Further examinations of the pelvis, for example by means of ultrasound, are not necessary.

It is advantageous if the planning data set comprises implantation information for an implant, in particular an artificial acetabulum, wherein the implantation information is adapted to the orientation of the patient during the treatment and are providable to the surgeon. In the planning data set, the orientation of the implant that is implanted when the patient is lying down can be adapted, in particular corresponding to the pelvic tilt when the patient is standing, in order to ensure a best possible implantation.

The planning data favorably comprise at least one parameter relevant to the operation on the bone, for example the location of a characteristic plane, a characteristic axis, and/or a characteristic point in relation to the bone in the planning data set.

The parameters that, in particular, may be considered to be clinically relevant may be visually superimposed, during the performance of the procedure, over the actual bone seen by the surgeon or depicted on the display device by means of images, for example by way of augmented reality. In a hip surgery, for example the plane of the acetabulum, the axis of the acetabulum, or the rotation center may be added to the planning data set as relevant parameters.

The data processing device is preferably configured and programmed to simulate the surgery outcome on the basis of the planning data and to provide information corresponding thereto to the surgeon. For example, as part of the simulation, the mechanical stability of the bone in the case of an implantation can be assessed. For this purpose, the data processing device may perform e.g. a finite element calculation on the basis of the planning data.

In a preferred embodiment, the instrumentation may comprise a storage unit in which information relating to previous treatments are stored, wherein the data processing device is advantageously configured and programmed to assess a treatment success based on the information and the planning data and to provide information corresponding thereto to the surgeon. Information about surgery outcomes for previous treatments, in particular over a longer period of time, can be stored in the storage unit. The data processing device can use this information including associated data (e.g. surgery parameters and classification of defects of the bone). The assessment of bone defects is based on quantitative determinations. Treatments to the bone and the successes thereof can be individually detected and stored. A large basis of data can advantageously be used, which, for example, may be classified according to bone defects. Modern methods like e.g. machine learning and neural networks enable, for example, an assessment as to whether a treatment will be successful according to probability, in the case of unknown bone defects or bone defects occurring for the first time. Corresponding indications can be provided to the surgeon in order to create a treatment strategy. For example, the data may show that a certain implant yields poor results above a certain defect size and instead a different treatment strategy should be chosen.

Information regarding the success of the treatment is advantageously suppliable to the storage unit for storage by way of an input interface, to be taken into account in later treatments. In this way, the database can be improved for later treatments and corresponding indications to the surgeon.

For detecting the characteristic landmarks of the bone intraoperatively, for example an ultrasound probe may be provided, to which a marking device that is detectable by the navigations system is fixed.

Alternatively or in addition, a palpation tool may be provided for the same purpose, to which a marking device that is detectable by the navigation system is fixed.

Provision may be made for the navigation system to comprise or form at least one data processing device or vice versa.

The creation of the actual state data set, the healthy state data set, and the planning data set may be performed, for example, by the same data processing device as used for the intraoperative use of the planning data set. Alternatively, provision may be made for the planning data to be transmitted to a different data processing device for intraoperative use.

At least two of the following tasks are performable by means of the same data processing device or by means of different data processing devices:
creating the actual state data set;
creating the healthy state data set;
creating the planning data set;
displaying the planning data set on the display device.

As mentioned at the outset, the present disclosure also relates to a method.

The object stated at the outset is achieved by a medical-technical method in accordance with the present disclosure, in which:

an actual state data set of a bone of a patient, in particular the human pelvic bone, which is considered to be defective, is created based on examination data;
a healthy state data set of the bone is created computationally on the basis of the actual state data set;
a planning data set of the bone is created on the basis of the healthy state data set and instructions of the surgeon regarding a treatment of the bone, which instructions are providable by way of an input device of the instrumentation, wherein the planning data set in particular comprises information about characteristic anatomical features of the bone;
wherein the planning data set is displayable on a display device;
wherein a marking device for defining a reference being fixed to the bone is detected by means of a medical-technical navigation system, and location and/or position data of the marking device are provided by the navigation system;
and the planning data set is displayed on the display device in spatial relationship with the bone, associating characteristic landmarks of the bone with corresponding characteristic landmarks in the planning data set.

The advantages which were already discussed in conjunction with the explanation of the instrumentation in accordance with the present disclosure can also be achieved in performing the method. Reference may be made to the preceding descriptions in this regard.

Advantageous embodiments of the method result from advantageous embodiments of the instrumentation in accordance with the present disclosure. Reference may be made to the preceding remarks in this regard.

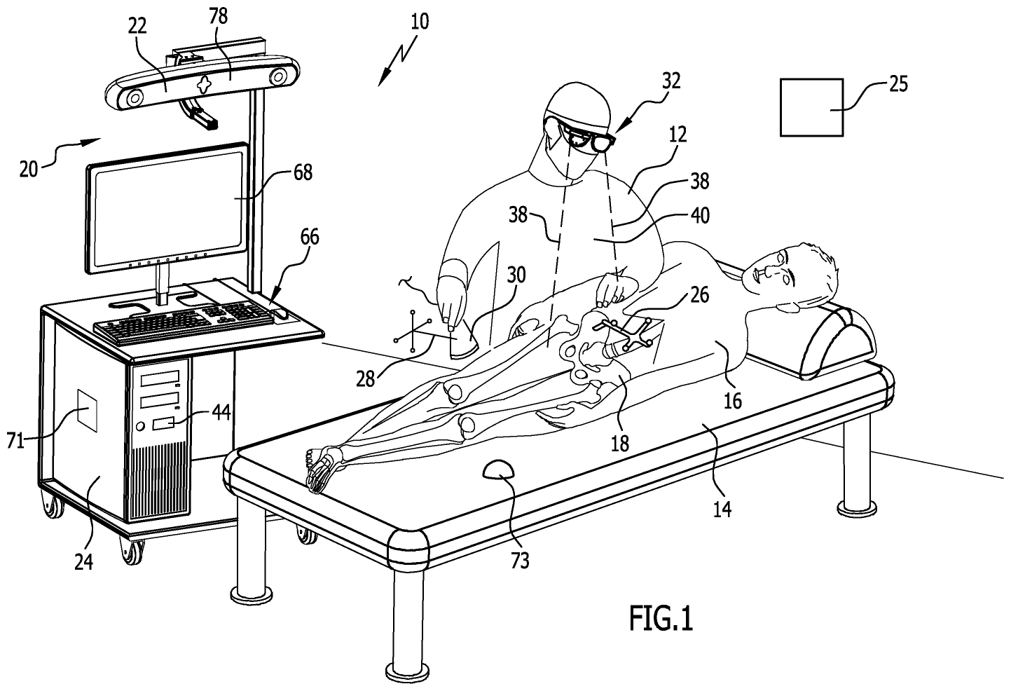

FIG. 1 shows in a schematic depiction an advantageous embodiment of an instrumentation in accordance with the present disclosure, attributed with the reference numeral 10. Moreover, a surgeon 12 is depicted treating a patient 16 lying on a patient support 14. The patient 16 undergoes a surgical procedure, in particular a revision surgery on a bone 18. The bone 18 is presently the pelvic bone.

The drawing accordingly shows the application of the present disclosure in a revision procedure in hip endoprosthetics. For example, an artificial acetabulum of the patient 16, which is not depicted in the drawing, is replaced by a new acetabulum, also not depicted. The bone 18 generally has bone defects that make the procedure more difficult for the surgeon 12. To make the procedure easier for a surgeon 12 with view to a better surgery outcome, the surgeon 12 may use the present disclosure.

The instrumentation 10 comprises a medical-technical navigation system 20 which, in the present embodiment, has an optical measuring system 22. The measuring system 22 is in operative connection with a data processing device 24 of the navigation system 20.

A medical-technical marking device 26 can be detected in a known manner by means of the measuring system 22. The data processing device 24 can transmit corresponding location and/or position data to the data processing device 24. It is possible, in particular, to track the marking device 26 in space.

In the present case, the marking device 26 is fixed to the bone 18, for example by screwing or adhesion, for defining a reference. Alternatively, a non-invasive attachment of a medical-technical marking device to the bone 18 is conceivable.

The instrumentation 10 also comprises a further marking device 28. The marking devices 26, 28 may be distinguished by the navigation system 20. The marking device 28 is presently held on an ultrasound probe 30. By means of the ultrasound probe 30, characteristic landmarks of the bone 18 can be detected in a non-invasive manner, and thereby be localized in the reference system of the marking device 26.

For example the posterior superior iliac spines and the pubic bone may be determined as characteristic landmarks of the bone 18 for defining the anterior pelvic plane in the reference system. The use of other or further characteristic landmarks that deliver information on the location and orientation of the bone 18 in space is of course also conceivable.

In place of the navigated ultrasound probe 30, e.g. a not depicted palpation tool with a probe tip on which a marking device is held may be used to palpate characteristic landmarks.

The marking devices 26, 28 may be passive and in particular retroreflective for radiation emitted from the measuring system 22. The use of active marking devices is also conceivable.

The instrumentation 10 further comprises a pair of data glasses 32 that can be worn by the surgeon 12. Alternatively or in addition, a head-mounted display, wearable on the head of the surgeon 12, may be provided, for example in the form of VR glasses.

The data glasses 32 presently have a conventional glasses frame with earpieces 34 for placing on the ears and a bridge 36 for placing on the nose of the surgeon 12. Provision may be made for the data glasses 32 to comprise optical lenses. However, this is not essential to the present disclosure.

The surgeon 12 can observe the scene through the data glasses 32. A region marked by dashed lines 38 in FIG. 1 symbolizes a field of vision 40 of the surgeon 12. The field vision 40 is presently directed toward the bone 18.

The data glasses 32 presently have a display device 42. The display device 42 is arranged on the data glasses 32 in such a way that image content on the display device 42 can be perceived by the surgeon 12 when naturally viewing a scene. The image content is therefore shown in such a way that it is located within the field vision 40. This makes it possible to display information to the surgeon 12 on the display device 42, which information can be superimposed over the scene viewed by said surgeon 12 in the sense of an augmented reality (AR).

For example the data processing device 24 is provided for providing the image content on the display device 42. For example, information relating to the image content is transmitted, preferably wirelessly, by the data processing device 24 via corresponding communication members 44, 46 to the data glasses 32.

It is preferably conceivable for the data glasses 32 to have an independent data processing device 48, which is in communicative connection with the data processing device 24, wherein the display device 42 can be controlled by the data processing device 48.

The data glasses 32 presently have a marking device 50 that is detectable by the measuring system 22. This offers the possibility of determining the location and orientation of the data glasses 32 by means of the navigation system 22. This makes it possible in particular to determine in which direction the field of vision 40 of the surgeon 12 is directed. As a result thereof, the field of vision 40 can be brought into relationship with the reference system defined by the marking device 26.

For example, if the surgeon is viewing the bone 18, said bone 18 is located in the field of vision 40. Augmented reality information relating to the bone 18 can be displayed on the display device 42 in such a way, as described in the following, as if it were on the bone 18 itself or in place of the bone 18. Alternatively or in addition, it is possible to display augmented reality information on the display device 42 in such a way that the surgeon 12 can perceive and “examine” it together with the bone 18, for example laterally next to the bone 18, above or below it etc.

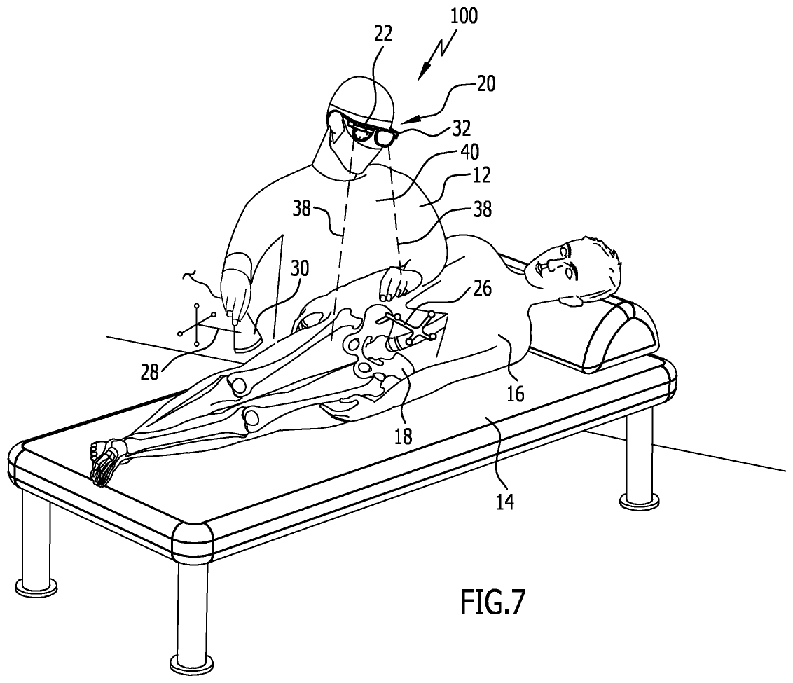

FIG. 7 shows an advantageous embodiment of the instrumentation in accordance with the present disclosure, designated with the reference numeral 100, in which the navigation system 20 is formed by the data glasses 32 and, unlike the instrumentation 10, no spatially separate navigation system 20 is present. The instrumentation 100 will still be described in the following.

A planning of the procedure can be performed preoperatively with the data processing device 24 or a further data processing device of the instrumentation 10 in accordance with the present disclosure. For this purpose, first, in particular, an actual state data set of the bone 18 can be created based on examination data. The actual state data set advantageously comprises a three-dimensional depiction of the bone 18.

The examination data are based, for example, on X-ray images or CT images. For this purpose, the instrumentation 10 may have at least one imaging device 25, depicted schematically in FIG. 1. A plurality of imaging devices 25 may be present.

The imaging device 25 may be e.g. an X-ray apparatus or a CT apparatus for preoperative and/or intraoperative use.

It may be advantageous if the pelvic tilt of the patient 16 can be determined from the combination of X-ray images and CT images. It is conceivable for the pelvic tilt to be determinable in a patient when standing and in a patient when lying down.

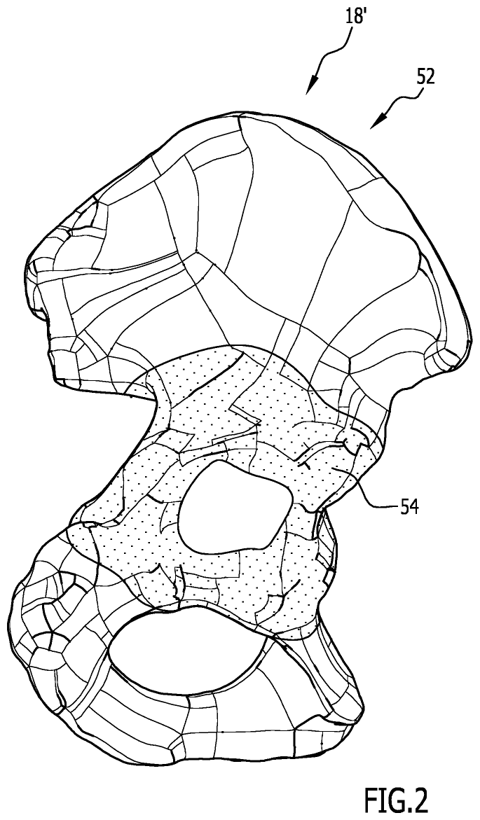

FIG. 2 shows in a view from lateral the image content of the actual state data set 52 of the bone designated here with the reference numeral 18′. The actual state data set preferably has, like the further data sets discussed above, a 3D representation of the bone.

In the actual state data set, the surgeon 12 can first visually determine the quality of the bone 18 and in particular assess the extent to which bone defects are present.

FIG. 2 identifies sections of the bone 18 with bone defects in an area 54 highlighted by means of points. It is understood that further areas 54 with bone defects are present on the actual state data set 52 and could be displayed, for example when said actual state data set 52 is examined in three dimensions.

The data processing device 24 is configured and programmed to computationally create a healthy state data set of the bone 18 on the basis of the actual state data set. Statistical models are used here. In particular the sex, age, weight, height, medical history and/or socio-cultural background of the patient 14 may also be taken into account.

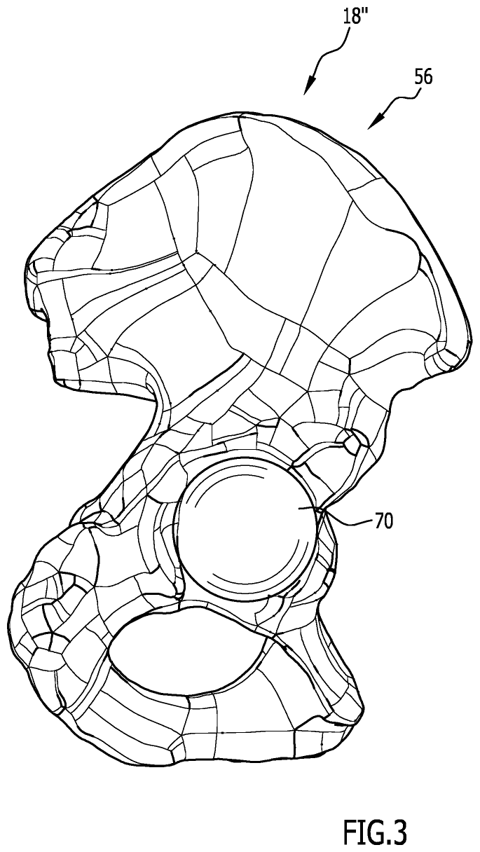

FIG. 3 shows a two-dimensional depiction of the healthy state data set 56 of the bone, designated here with the reference numeral 18″, in a view from lateral.

It is advantageous for the healthy state data set 56 to be calculated individually to the patient and for a generic data set to not have to be used.

The data processing device 24 is configured and programmed such that it can computationally determine deviations between the actual state data set 52 and the healthy state data set 56. In particular, a classification of the deviations can thereby be performed, for example with respect to bone loss, bone growth, material different from bone such as bone replacement material or bone cement, the amount or degree of the deviation as well as the location of the deviation.

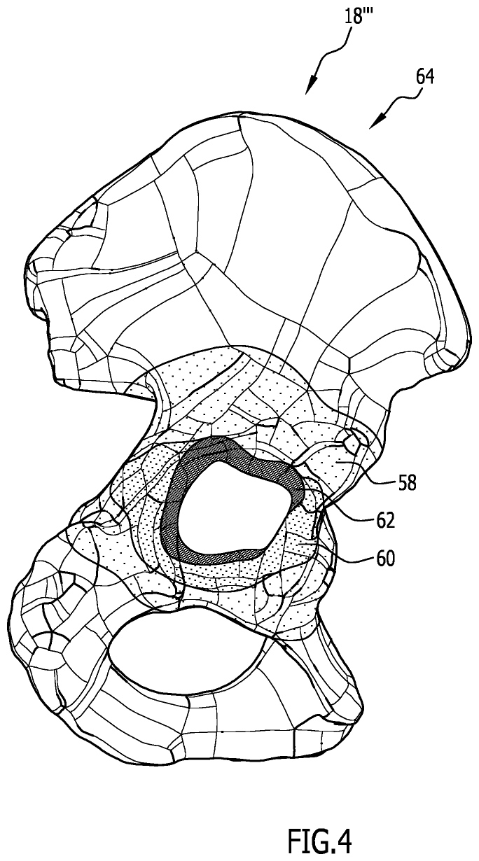

The classification of the deviations can be performed on the bone 18, for example in segments, by the data processing device 24. For example, the bone 18 is computationally subdivided into clinically relevant sectors. FIG. 4 schematically depicts this with the example of sectors 58, 60, and 62, which are each highlighted differently in the graphic representation.

FIG. 4 depicts a planning data set 64 created in the planning for the operation with viewing direction from lateral to the bone presently designated with the reference numeral 18″. For example the bone defects of sectors 58, 60, 62 determined on the basis of the classification are taken into account for creating the planning data set 64. In particular, it is possible for the surgeon to carry out the planning using an input device 66 of the instrumentation 10. The surgeon 12 can thereby use, adapt, and evaluate the planning data set 64 with a view to an optimal implantation outcome.

The data processing device 24 can present the surgeon 12, for example, indications regarding the implantation technique and/or the implant selection, in dependence on the classification of the bone defects. The indications can be added to the planning data set 64 and/or displayed on a display device 68 shown as an example.

It is also conceivable, in particular, for relevant parameters that are of importance to the procedure to be added to the planning data set 64. Relevant parameters are e.g. the location of the plane of the acetabulum 70, the location of the axis of the acetabulum 70, and/or the location of the rotation center. The surgeon 12 can preoperatively interact with the instrumentation in accordance with the present disclosure in order to, for example, check said relevant parameters in the planning data set 64, or the data processing device 64 can make appropriate suggestions.

The instrumentation may comprise a storage unit 71, which is presently integrated into the data processing device 24. The storage unit 71 may also be arranged spatially separated from the data processing device 24 and be coupled thereto.

Information regarding earlier similar treatments are advantageously stored in the storage unit 71, which information can be taken into account by the data processing device 24 in order to assess a treatment success based on the planning data set 64. Corresponding information can be provided to the surgeon 12, for example by way of the display device 68.

The storage unit 71 may comprise an input interface, for example by way of the input device 66, for supplying information relating to the success of the treatment. Information of that kind can be taken into account in later treatments for creating the planning data sets.

It may be advantageous if the planning data set 64 comprises implantation information for an implant 73, in the present case an artificial acetabulum. It is thereby favorable if information about a previously determined pelvic tilt of the patient 16 is present in the planning data set. For example, the planning data set takes into account the information on the pelvic tilt, adapted to the surgical situation. It can thereby be taken into account, for example, that the patient 16 is lying down during the operation. Implantation information for aligning the implant 73 may be adapted to the lying state of the patient, it being taken into account that by means of this adaptation and taking into account the pelvic tilt when the patient is standing and the pelvis is bearing load, a best possible implantation with a view to stability and range of motion exists.

For intraoperative use, the planning data set 64 can, if necessary, be transmitted to a data processing device in the operating room. Otherwise, the planning data set 64 created on the data processing device 24 can be used.

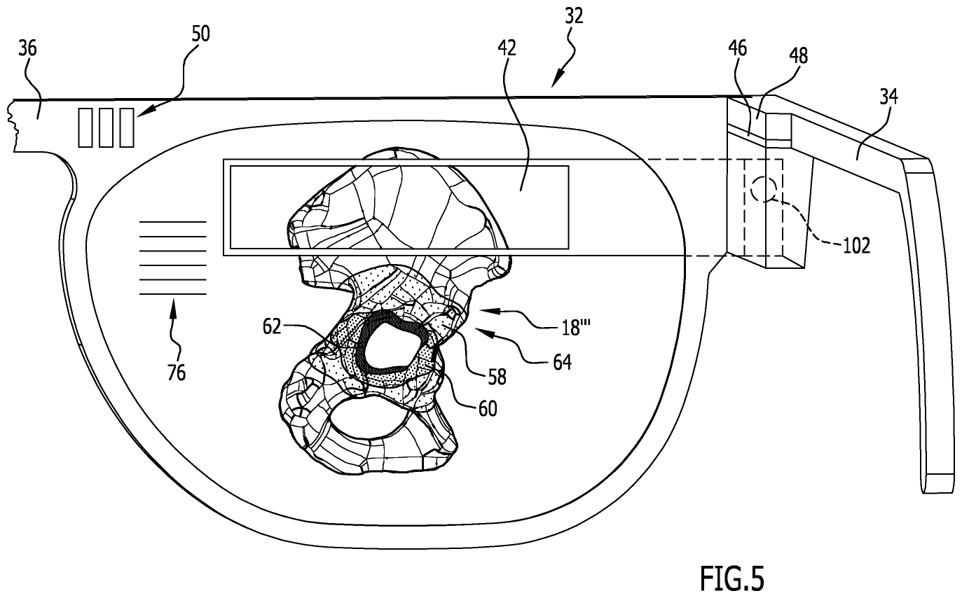

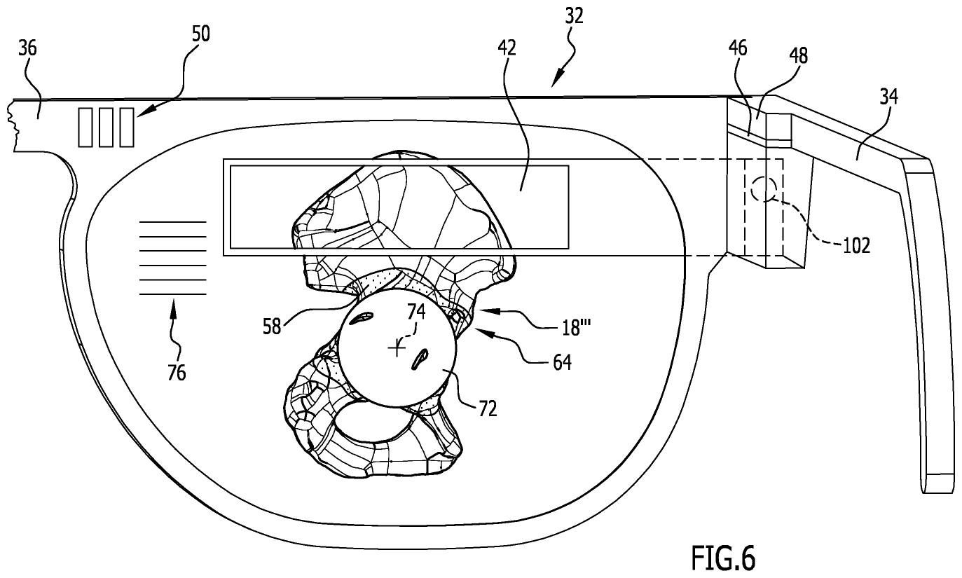

The surgeon 12 can, as described, determine characteristic landmarks on the bone 18, the locations of which can be determined in the reference system of the marking device 26. The gaze of the surgeon 12 is directed e.g. at the bone 18, detectable by the navigation system 20. Image content of the planning data set 64 can be displayed in the real scene observed by the surgeon 12 in order to assist the surgeon 12 during the procedure. This is schematically depicted in FIGS. 5 and 6.

The association of the characteristic landmarks of the real bone 18 with the corresponding characteristic landmarks of the bone 18′″ in the planning data set 64 can be made computationally by the data processing device 24. The spatial information contained in the planning data set 64 is thereby brought into conformity with the real geometry of the bone 18 and set in defined spatial relationship thereto.

The association of characteristic landmarks of the bone 18 with the actual state data set 52 and/or with the planning data set 64 can take place e.g. intraoperatively using the imaging device 25. For example, a C-arc X-ray apparatus, which generates an X-ray image of the bone 18 together with the marking device 28, is used as the device 25. As already explained at the outset, the 2D X-ray image can be superimposed over the 3D model. A referencing of the physical bone 18 relative to the 3D model can be created by way of the marking device 28.

In addition, further information in the planning data set 64 can be displayed on the display device 42. FIG. 5 depicts this exemplarily for the different bone defects in sectors 58, 60, and 62. FIG. 6 schematically shows an example of the location of the plane 72 of the acetabulum 70 and its axis 74. FIGS. 5 and 6 symbolize further contents, designated with reference numeral 76, of the planning data set 64, configured as instructions 76 for instructing the surgeon during the procedure.

The surgeon 12 can use the additional information in augmented reality and compare the real situation during the procedure with the planning done in advance with a view to an optimal surgical outcome.

The display device 42 is preferably updated in real time, such that the image content of the display device 42 can always be displayed in the correct position upon a movement of the surgeon 12 and/or the patient 14.

It is understood that the depiction in FIGS. 5 and 6 is merely schematic. The display of the planning data set 64 and the instructions 76 is perceived by the surgeon 12 as if the depicted image content were located at the correct normal position within the surgeon's field of vision 40, with the surgeon 12 merely looking through the display device 42.

The aforementioned advantageous embodiment of the instrumentation 100 in FIG. 7 does not have an external navigation system 20. In particular, the navigation system 20 is integrated into the data glasses 32 or is formed thereby. It is thus not necessary to track the data glasses 32, provided with the marking device 50, by means of a navigation system.

For detecting the marking devices 26, 28 the data glasses 32 in the instrumentation 100 have, in particular, an integrated measuring system with a camera 102, which is used in place of the navigation camera 78 of the measuring system 22.

In all other respects, the advantages achievable by means of the instrumentation 10 can also be achieved with the instrumentation 100, such that reference may be made to the above statements in this regard.

In a further preferred embodiment, an integrated, hand-holdable navigation system may be provided, for example in the form of a smartphone or a tablet computer. It may comprise a camera for detecting the scene, wherein images of the scene can be displayed on a display device. The information of the planning data set 64 may augment the image content of the images and be used in a corresponding manner to the instrumentations 10, 100.

1. Medical-technical instrumentation comprising at least one data processing device, which is configured and programmed to create an actual state data set of a bone that is considered to be defective of a patient, based on examination data;
to create computationally a healthy state data set of the bone on the basis of the actual state data set;
to create a planning data set of the bone on the basis of the healthy state data set and of instructions of the surgeon regarding a treatment of the bone, which instructions are providable by way of an input device of the instrumentation, wherein the planning data set comprises information about characteristic anatomical features of the bone;
to display the planning data set on a display device of the instrumentation;
wherein the instrumentation comprises a medical-technical navigation system and a marking device for defining a reference, which marking device is detectable by said navigation system and is fixed or fixable to the bone, wherein at least one of location and position data of the marking device are providable by the navigation system;
and wherein the planning data set is displayable by at least one data processing device on the display device in spatial relationship to the bone, associating characteristic landmarks of the bone with corresponding characteristic landmarks in the planning data set.

2. The medical-technical instrumentation according to claim 1, wherein at least one of the following applies:
image content of the planning data set is displayable on the display device in real time;
the navigation system comprises at least one camera for detecting the bone and images corresponding thereto are displayable on the display device in combination with the display of the planning data set;
the actual state data set comprises a three-dimensional representation of the bone.

3. The medical-technical instrumentation according to claim 1, comprising at least one imaging device, in the use of which at least one of the following is createable:
the actual state data set;
an auxiliary data set, by means of which the association of the characteristic landmarks is performed by the data processing device, wherein the marking device is registered by the data processing device relative to at least one of the actual state data set and of the planning data set.

4. The medical-technical instrumentation according to claim 3, wherein at least one of the following applies:
the at least one imaging device is or comprises an X-ray apparatus or a CT apparatus;
the auxiliary data set is or comprises an X-ray image of the bone and the marking device, and wherein the data processing device superimposes the X-ray image over at least one of the actual state data set and of the planning data set.

5. The medical-technical instrumentation according to claim 1, wherein the marking device is fixed or fixable to the bone at least one of directly or indirectly and in a non-invasive or invasive manner.

6. The medical-technical instrumentation according to claim 1, wherein the navigation system comprises a head-mounted display or a pair of data glasses, which comprises the display device.

7. The medical-technical instrumentation according to claim 1, wherein at least one of the following applies:
a marking device, which is detectable by the navigation system, is arranged on the display or on the data glasses, the movement of said marking device in space being tracked, wherein the display of the planning data set takes place in dependence on at least one of the location and the orientation of the display or data glasses;
the display or the data glasses form the navigation system for detecting the marking device on the bone;
the instrumentation comprises a hand-holdable integrated navigation system, which comprises at least one data processing device and the display device.

8. The medical-technical instrumentation according to claim 1, wherein the data processing device is configured and programmed to determine deviations between the actual state data set and the healthy state data set.

9. The medical-technical instrumentation according to claim 8, wherein the data processing device is configured and programmed to classify deviations with respect to at least one of the following:
bone loss;
bone growth;
material different from bone, for example bone replacement material or bone cement;
amount or degree of deviation;
location of the deviation.

10. The medical-technical instrumentation according to claim 9, wherein the data processing device is configured and programmed to perform classification of the bone in segments.

11. The medical-technical instrumentation according to claim 10, wherein the data processing device is configured and programmed to, in dependence on the classification, provide indications on the display device, and/or add indications to the planning data set, relating to at least one of the following for the surgeon:
indications regarding implantation technique;
indications regarding implant selection.

12. The medical-technical instrumentation according to claim 1, wherein in a treatment of the pelvic bone, the planning data set comprises information about the pelvic tilt when the patient is standing or is lying down and/or wherein the planning data set comprises implantation information for an implant, which information is adapted to the orientation of the patient during the treatment and is providable to the surgeon.

13. The medical-technical instrumentation according to claim 1, wherein the planning data set comprises at least one parameter relevant to the operation on the bone, for example the location of a characteristic plane, a characteristic axis, and/or a characteristic point in relation to the bone in the planning data set.

14. The medical-technical instrumentation according to claim 13, wherein the parameters are displayable on the display device by way of augmented reality and are visually superimposed over the bone viewed by the surgeon or displayed on the display device by means of images.

15. The medical-technical instrumentation according to claim 1, wherein at least one of the following applies:
the data processing device is configured and programmed to simulate the surgery outcome on the basis of the planning data and to provide the surgeon with information corresponding thereto, in particular with a view to the mechanical stability of the bone in the case of an implantation;
the instrumentation comprises a storage unit in which information relating to previous treatments is stored, wherein the data processing device is configured and programmed to assess a treatment success based on the information and the planning data and to provide information corresponding thereto to the surgeon.

16. The medical-technical instrumentation according to claim 15, wherein information relating to the success of the treatment is suppliable to the storage unit by way of an input interface for storage, to be taken into account in later treatments.

17. The medical-technical instrumentation according to claim 1, wherein the instrumentation, for detecting the characteristic landmarks, comprises at least one of the following:
an ultrasound probe, to which a marking device that is detectable by the navigation system is fixed;
a palpation tool, to which a marking device that is detectable by the navigation system is fixed.

18. The medical-technical instrumentation according to claim 1, wherein the navigation system comprises or forms at least one data processing device or vice versa.

19. The medical-technical instrumentation according to claim 1, wherein at least two of the following are performable by means of the same data processing device or by means of different data processing devices:
creating the actual state data set;
creating the healthy state data set;
creating the planning data set;
displaying the planning data set on the display device.

20. A medical-technical method comprising the following steps:
creating an actual state data set of a bone of a patient, which bone is considered to be defective, based on examination data;
creating a healthy state data set of the bone computationally based on the actual state data set; and
creating a planning data set of the bone based on the healthy state data set, the anatomical characteristics and instructions of the surgeon regarding a treatment of the bone, which instructions are providable by way of an input device of instrumentation,
wherein the planning data set comprises information about characteristic anatomical features of the bone,
wherein the planning data set is displayable on a display device,
wherein a marking device for defining a reference being fixed to the bone is detected by a medical-technical navigation system,
wherein at least one of location and position data of the marking device are provided by the navigation system, and
wherein the planning data set is displayed on the display device in spatial relationship with the bone, associating characteristic landmarks of the bone with corresponding characteristic landmarks in the planning data set.
